(19) United States
(12) Patent Application Publication
(10) Pub. No.: US 2020/0072736 A1
(43) Pub. Date: Mar. 5, 2020

(54) IR Probe for Detection of Contaminants in Sealed Containers

(71) Applicant: Stuart B. Long, Camarillo, CA (US)

(72) Inventors: Stuart B. Long, Camarillo, CA (US); Damon Brink, Ojai, CA (US)

(21) Appl. No.: 16/553,097

(22) Filed: Aug. 27, 2019

Related U.S. Application Data
(60) Provisional application No. 62/724,352, filed on Aug. 29, 2018.

(51) Int. Cl. G01N 21/31; G01N 21/90

(52) U.S. Cl. CPC G01N 21/314; G01N 33/94; G01N 21/90

(57) ABSTRACT

Systems and processes for which containers are placed to detect contamination more effectively than current practices may be provided. This may be done using a combination of imaging techniques. Specifically, one approach combines UV, visible, and IR images to enhance the contrast of contamination in sealed pharmaceutical containers. The images may be further enhanced through the use of a flash of heat. The use of resonance to manipulate the vial contents may be further used. These approaches may be combined with mechanical motion so that the material inside the container is turned over in a way to expose the potential contamination. In some embodiments the orientation of the sealed container may be positioned to maximize the surface area and inspection volume of the material under investigation.

### RELATED APPLICATIONS

This application claims the benefit of priority to U.S. Provisional Application 62/724,352 filed Aug. 29, 2018, and hereby incorporated by reference in its entirety.

### BACKGROUND

This disclosure relates to pharmaceutical packaging and in particular to the reduction of contaminants in pharmaceutical packaging.

Pharmaceuticals are packaged in sterile environments and subject to strict Good Manufacturing Practice (GMP) requirements. Nevertheless, contamination can often be found inside the sealed vials and drug companies must inspect the containers and contents to guard against contamination. New techniques and equipment to streamline the inspection process may be desirable.

### SUMMARY

Systems and processes for which containers are placed to detect contamination more effectively than current practices may be provided. This may be done using a combination of imaging techniques. Specifically, one approach combines UV, visible, and IR images to enhance the contrast of contamination in sealed pharmaceutical containers. The images may be further enhanced through the use of a flash of heat. The use of resonance to manipulate the vial contents may be further used. These approaches may be combined with mechanical motion so that the material inside the container is turned over in a way to expose the potential contamination. In some embodiments the orientation of the sealed container may be positioned to maximize the surface area and inspection volume of the material under investigation.

### BRIEF DESCRIPTION OF THE DRAWINGS

Aspects and advantages of the embodiments provided herein are described with reference to the following detailed description in conjunction with the accompanying drawings. Throughout the drawings, reference numbers may be re-used to indicate correspondence between referenced elements. The drawings are provided to illustrate example embodiments described herein and are not intended to limit the scope of the disclosure.

FIG. 1 illustrates an embodiment of a Process Flow Diagram

FIG. 2 illustrates an embodiment of a Test Sequence Process Flow

FIG. 3 illustrates an embodiment of a vial and its components.

FIG. 4 illustrates an embodiment of a syringe and its components.

FIGS. 5A and 5B illustrate an embodiment of a the Head unit with various components in location.

FIGS. 6A and 6B illustrate an embodiment of a the machine with vial in position.

FIG. 7 illustrates an embodiment of a the entire body with Head Unit and vial in position.

FIGS. 8A and 8B illustrate an embodiment of a Vial with Camera and LED combination

FIGS. 9A and 9B illustrate an embodiment of a Multiple cameras, multiple LED various positions.

FIG. 10 illustrates an embodiment of a the use of filters in use with LED's and cameras.

FIG. 11 illustrates an embodiment of a the use of multiple cameras, filters, LED's, Heat sources

FIG. 12 illustrates an embodiment of a Focus Lens, 2×2

FIG. 13 illustrates an embodiment of a Focus Lens, 2×4

FIG. 14 illustrates an embodiment of a Multi Detection module

FIG. 15 illustrates an embodiment of a Magnification implementation

FIG. 16: illustrates an embodiment of a Single heat source, single detector.

FIGS. 17A and 17B illustrate an embodiment of a Multi-heat source

FIG. 18 illustrates an embodiment of a Resonance chamber

### DETAILED DESCRIPTION OF ILLUSTRATIVE EMBODIMENTS

In some embodiments, a system may comprise a bench-top unit, comprised of a main housing, a form-factor insert, and a head-unit. The form-factor insert and head units can be changed and tailored to specific configurations for specific tasks such as testing for a specific drug in a particular syringe. The main housing may contain the power supply, drive motors, and electronics such as a PLC for programming different recipes for various manufacturing lines. Based on the recipe, the drive motors interface with the form-factor insert and move the container of pharmaceutical in a specific manner. Real time images as well as stand-alone images may be acquired of the drug/container combination using Infra-Red (IR), Visible, Ultra Violet, and Ultra Violet-Fluorescence imaging. The images may be taken during or after exposing the sample to heat in the infrared spectrum. The images may be taken during resonance excitation in combination with the heat flash. These images in some embodiments may be taken simultaneously and compared to a data base of good/bad images created for a specific drug/container combination. The images taken with different wavelength detectors may be superimposed to enhance contrast and eliminate false positives.

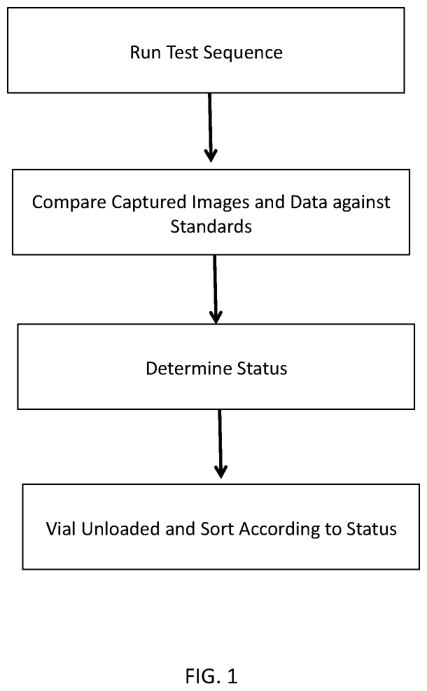

In some embodiments, an operator may place a vial by hand. The specific recipe may be input, and normally used for a large number of inspections. The inspector then initiates the test sequence, according to FIG. 1. In the process embodiment of FIG. 1, the container is normally placed horizontally. The machine then rotates the drug/container combination and takes images for comparison against the data base. Based on the comparison with the data base, the unit makes a decision as to whether or not contamination is identified in the container.

Pharmaceuticals may be packaged in a variety of containers with different form factors, including vials, syringes, and bags. The drugs can be classified by the physical state at the time of packaging, which include liquids, dry powders, and creams.

Types of contamination include stainless steel, aluminum, plastic, rubber, glass, and organic materials, as well as others and may come in a wide variety of shapes and sizes.

The packaged drugs may be inspected using visible light by camera systems and/or manual inspectors. For manual inspection, the inspections often shake the container and look at it on different background and sometimes different lighting conditions.

Compositional contrast achievable with visible light may be limited for some applications. Pharmaceutical manufacturers and contract manufacturers may perform sterile filling of vial, syringes, and other containers. The lack of contrast lowers the likelihood that a foreign contaminant will be detected and increases inspection time. Inspection methods and techniques that increase contrast and improve detection of foreign contaminants in sterile packed pharmaceuticals may be beneficial for some applications.

FIG. 1 is Process flow Diagram presenting the steps involved in using equipment in an exemplary embodiment.

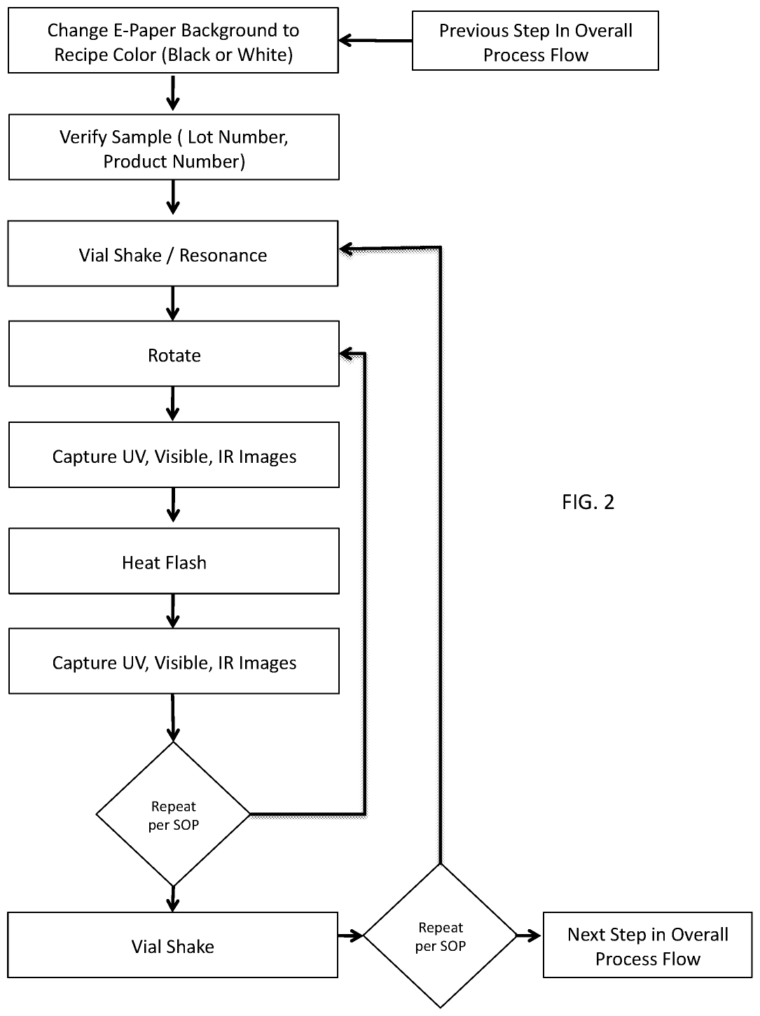

FIG. 2 is a Test Sequence Process Flow—Combined Illumination of Containers to Image Contamination in an exemplary embodiment

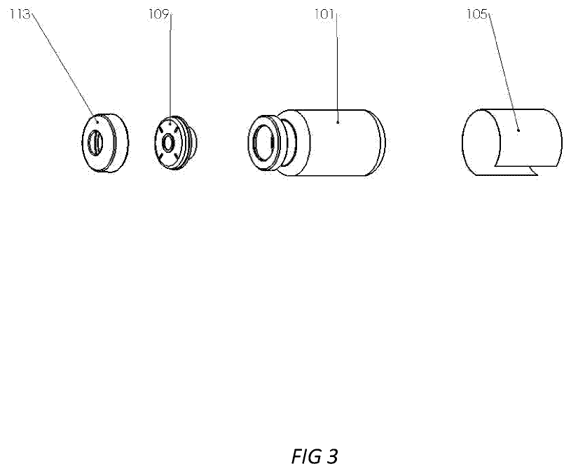

FIG. 3 shows the components of a vial. (113) Cap, (109) septum, (101) vial and (105) label. Vials in manufacturing can vary in size. After the manufacturing process vials and caps are wrapped with a shrink wrap plastic cover. (105) is not always on vial at time of inspection.

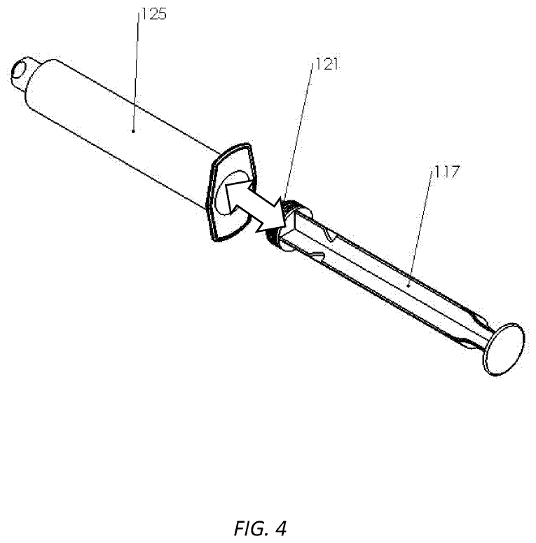

FIG. 4 shows the components of a syringe. (125) body, (121) septum and (117) plunger. Syringes can vary in size in manufacturing. The enclosure cap that is placed on the body vary.

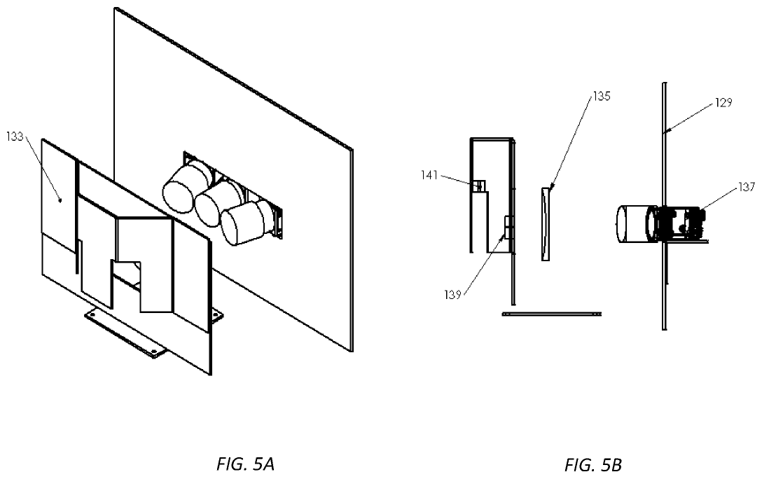

FIGS. 5A and 5B show an exemplary embodiment of the Head Unit. The Head Unit may include (129) mounting, (133) LED Frame, (135) Magnification, (137) various cameras, (139) Laser diodes, (141) LED's, filters, apertures, mounting devices, focus lens. The Head Unit will be assembled in various configurations. These configurations may be configured to accommodate the size of vials and syringes. The head unit may mount onto or into the Main unit. Cameras and other equipment will be mounted around the horizontally, angularly or vertically placed vial or syringe. The primary goal of mounting the cameras will be to have them in proximity of one another so the images will cover substantially the same Field of View (FOV).

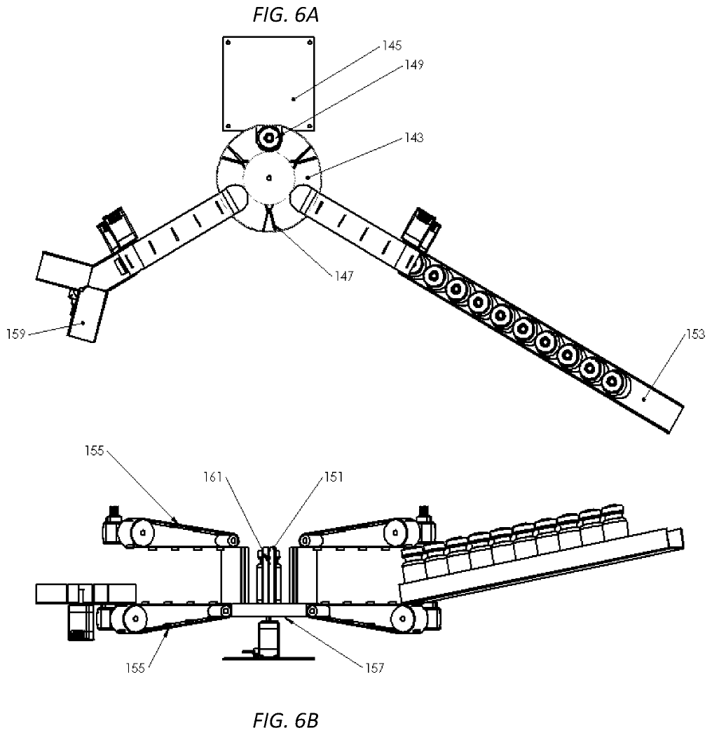

FIGS. 6A and 6B show an exemplary embodiment for a drive system with a vial mounted. This system embodiment may be (155) belt driven and controlled by various servo motors. The (149) vial could be mounted in a vertical, angular or horizontal orientation to optimize the viewing area of the contents while decreasing the depth of the material in the vial. While reflective mirrors may be used to view the cap and bottom surface. The vial may be held in place by a fitted end cap on one end. On the other end will be another fitted cap as well as a compression system (161) that will adjust for varying lengths of vials. From this orientation the motion control system will have the capability to rotate the vial plus or minus any degree deemed necessary. For dry sub-straights a technique has been created by rotating the vial some degree and capturing an image and then rotating again so the contents of the vial will have cause to fold over on itself exposing more of the contents with each folding. This motion control system will also be able to rotate the vial rapidly causing the contents to take flight inside the vial, creating a dust cloud, exposing the material in its thinnest state. This dust cloud approach will allow the camera system to essentially see through the cloud.

There could be several different configurations of head units depending on the user's needs.

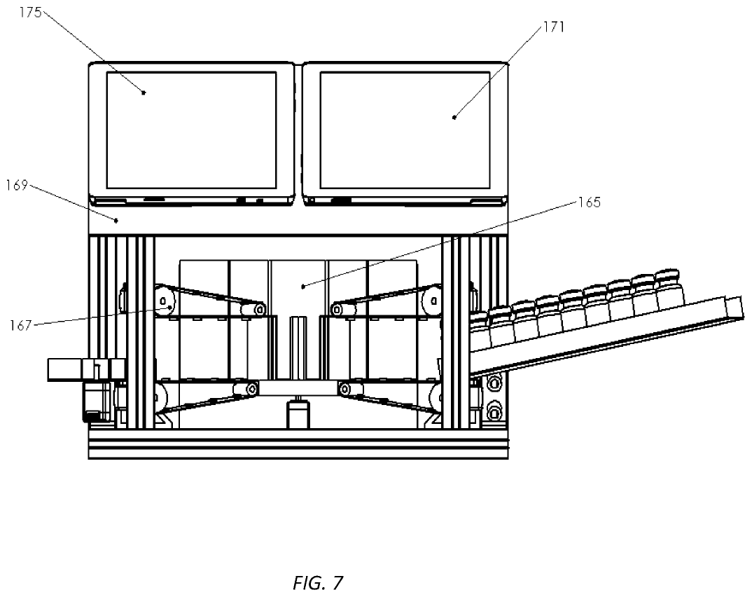

FIG. 7 shows an exemplary embodiment of the machine with the head unit (165) in position on the body (169) with primary (171) and secondary e=paper module (175) and the drive system (167). This approach will allow the user to swap out head units conveniently. In some embodiments two head units may be positioned on the system. The Head Unit imaging system will face the e-paper module. The e-paper module can change backgrounds that are color correct without having to move the vial. These backgrounds are used to enhance the difference in contrast. The secondary e-paper screen could be set up as a secondary inspection for vials that have suspected contamination. This approach would have the suspected vial divert from the line and then be inspected via screen or removed for hand inspection.

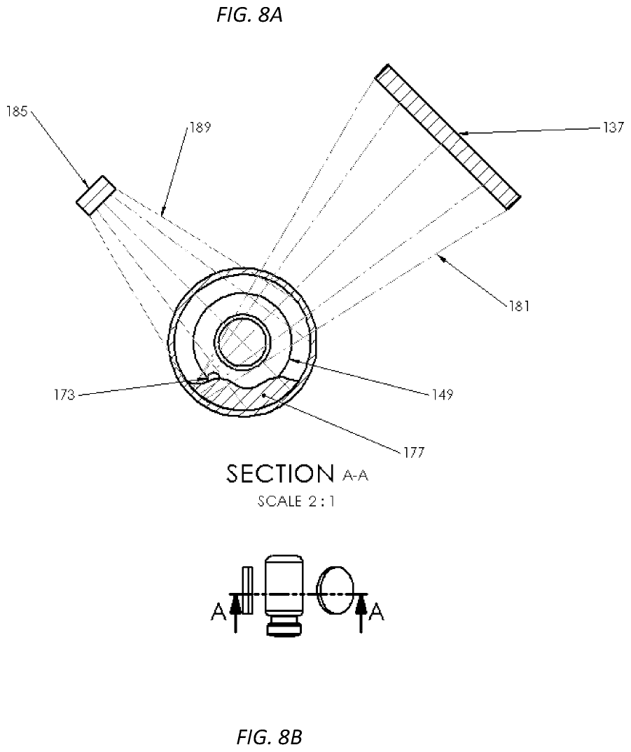

FIGS. 8A and 8B show a vial with contamination and one or more cameras in an exemplary embodiment. The camera (137) could be a SWIR: short wave infra-red camera, NIR; near infra-red camera, UV; ultraviolet camera, or visible light camera. SWIR imaging range may be between 0.9 and 1.7 um. This range can extend as low as 550 nm to as high as 2.5 nm. NIR typically operates in the range of 780 nm to 2500 nm. The UV cameras operate in a range 1 nm to 400 nm. This range can be broken in to 3 bands. Near UV operates in 380 to 200 nm, Far UV operates 200 nm to 10 nm and Extreme UV operates in 1-31 nm. The visible light cameras may operate in a range of 400 to 750 nm. The LED (185) could be a single LED or a bank of LED in 2×2, 2×3, up to 2×6 configuration, or other configurations. The LED's colors may include blue, green and or white. The Blue may operate at approximately 470 nm, the green may operate at approximately 560 nm and the white may operate in a range of 474 terra hertz and 638 terra hertz. These could be used individually or in any combination thereof. The LED's may be configured so that the substrate will not be irradiated with damaging lower wavelength spectra. The relative location of the LED's may present different opportunities with the backgrounds and other cameras and filters. (181) presents the focus of the camera and where it will be focusing on. (189) shows the spread of the LED light.

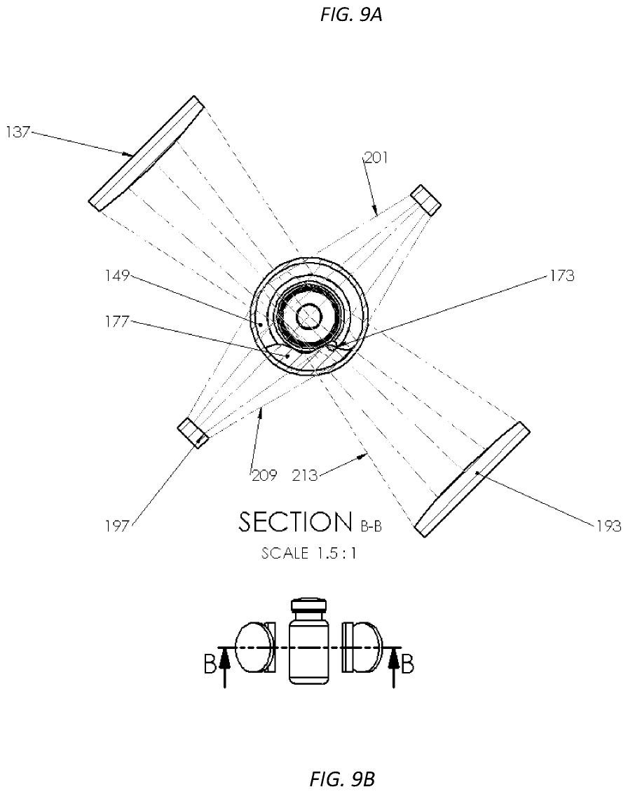

FIGS. 9A and 9B present an exemplary embodiment of a multi camera multi LED configuration. LED's (197) and cameras (193) could be placed in various configurations around the vial to increase the contrast of contamination to substrate. With the multiple cameras being simultaneous pictures may be acquired from each type of camera. This will provide up to 4 different images that can be compared to each other. Once the images are taken the vial will rotate approximately 30 degrees and then the images will be repeated. Having the vial rotate 30 degrees will allow enough movement in the substrate to cause it to fold over inside the vial. Exposing a different amount of substrate, that would once again be image captured. In some embodiments, some of these images may be taken from substantially the same location and distance allowing the images to be compared and contrasted to the previous set. Angles can be adjusted so more or less images are taken for each vial. Images may then be compared and saved and used in an image recognition software where contaminates and other issues can be identified. A library of images will be created and used as a base for the compare and contrast of images.

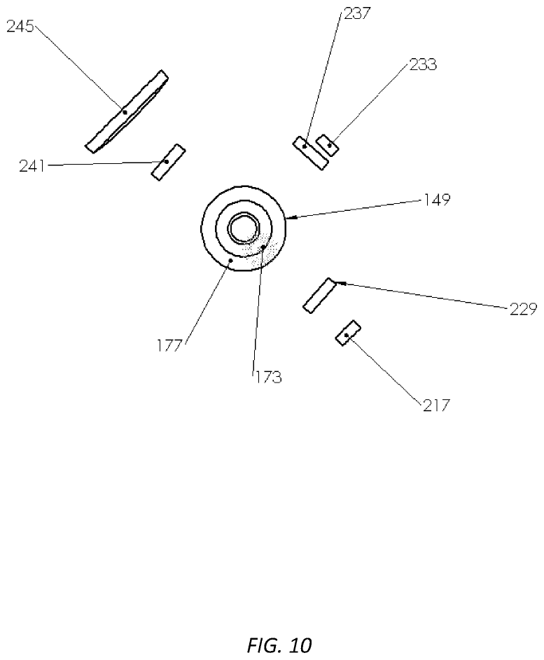

Referring to FIG. 10, in some embodiments, the use of optical filters to selectively transmit light and different wavelengths for various applications may be part of the system. Filters come in many different materials: glass or plastic, sizes and coatings and are used to enhance colors, reduce reflections and block longer or shorter wavelengths. The filter can be used to modify the light before it is applied to the vial.

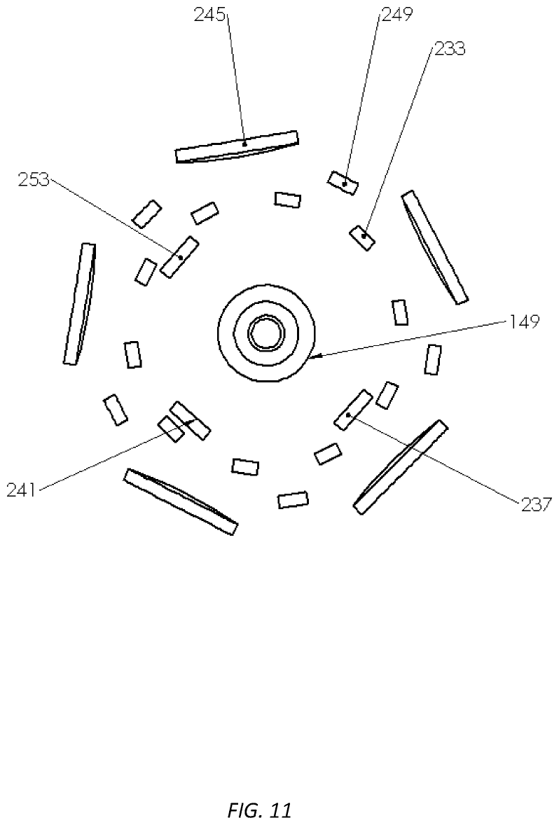

Referring to FIG. 11, in this embodiment there is representation of multiple LED banks (233), multiple LED filters (237), multiple camera filters (241), multiple cameras (245), These can be radially placed or linearly. Each item can be used individually or sequentially.

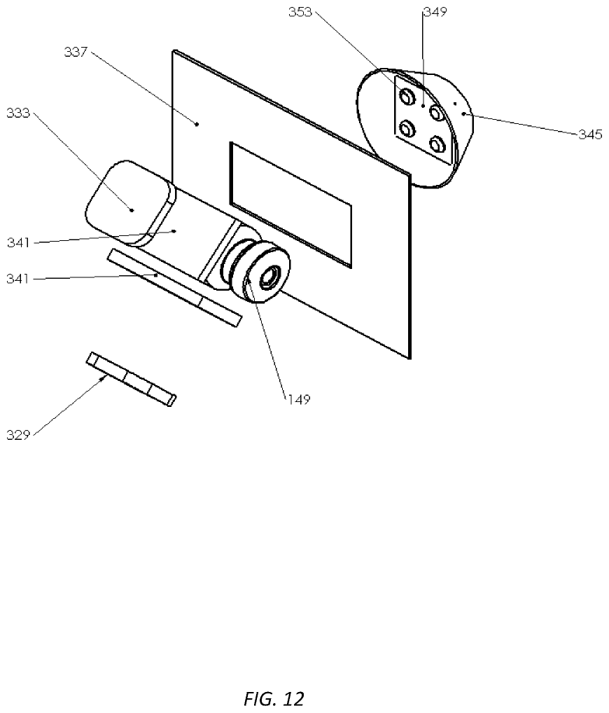

Referring to FIG. 12, in this embodiment there is a LED bank comprised of a PCB (349), LED's (353), and a shroud (345), an aperture (337), various types of cameras (329, 333) and magnification (341). The use of magnification between the cameras and the vial may allow objects in the field of vision to be increased in size and be more detectable. Magnification ranges may include 1× up to 10×. The magnification glass may be shaped to improve the camera image. The LED's (353) in this embodiment are presented in a 2×2 layout, but other layouts are possible. The LED's could be different wavelengths therefore causing a different light source to be reflecting a different emissivity. The LED's could be illuminated all at once or in a series or in a sequence.

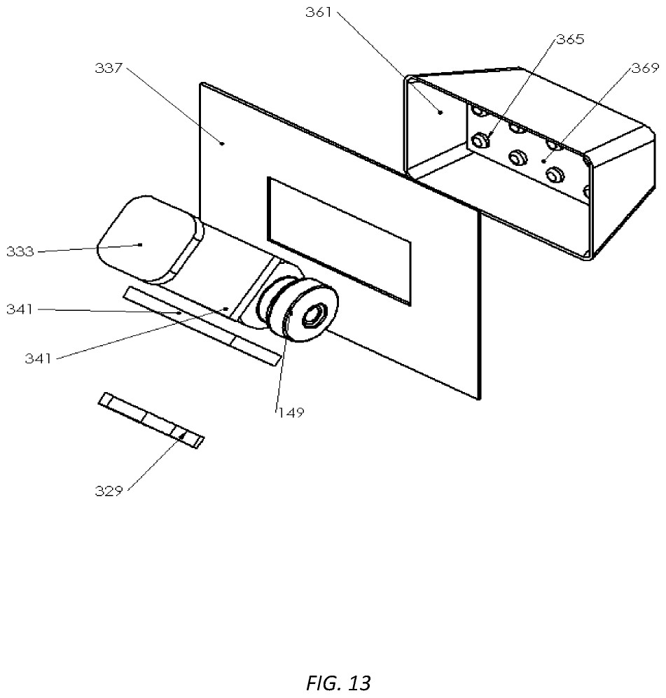

Referring to FIG. 13, in this embodiment there is a LED bank comprised of a PCB (369), LED's (365), and a shroud (361), an aperture (337), various types of cameras (329, 333) and magnification (341). The use of magnification between the cameras and the vial may allow objects in the field of vision to be increased in size and be more detectable. The magnification ranges could include 1× up to 10×. The magnification glass may be shaped to improve the camera image. The LED's (365) are presented in a 2×4 layout, other layouts are possible. The LED layout could be a 2×2 up to 2×6, or others. The LED's could be different wavelengths therefore causing a different light source to be reflecting a different emissivity. The LED's could be illuminated all at once, in a series or in a sequence.

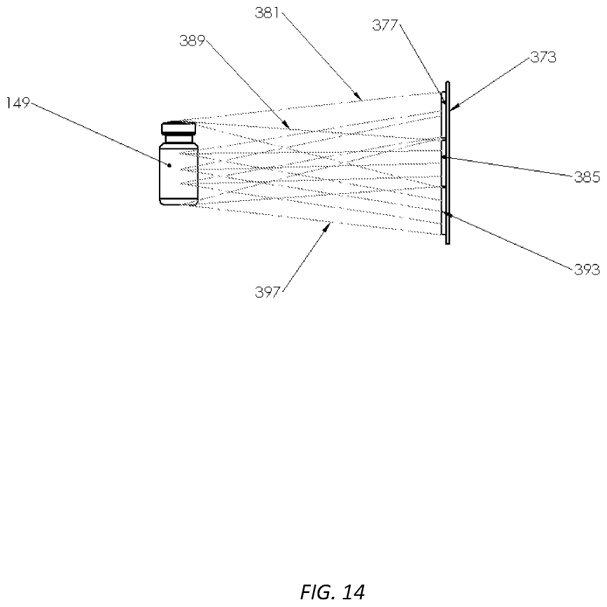

Referring to FIG. 14, in this embodiment there is a camera bank (373) with different cameras (377, 385, 393) that project onto the vial. In this embodiment, the camera angles may vary, up to 20 degrees. The objective is to have each camera be able to take substantially the exact same image, or at least a common imaged area as the other. Each image will be digitally overlaid with each other image to compare and contrast the differences. Optical adjustment could be used to remove camera angle.

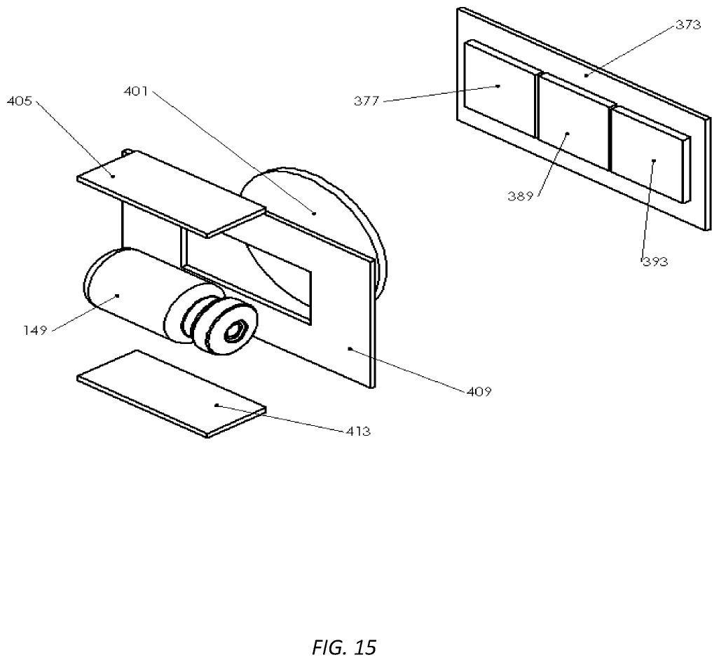

Referring to FIG. 15, in this embodiment there is a camera bank arrangement (373, 377, 389, 393) Each of these cameras represent a SWIR, NIR, UV or regular light camera. Their position is optically corrected so each camera is images at least part of the same FOV. (401) is a magnification lens used to enlarge the vial and its contents, Magnification range may include 1× and 10×. (409) is an aperture used to crop the camera image. (405, 413) are LED banks. The vial will be captured on either end and will rotate up to 30 degrees, after each rotation or during the rotation there will be a set of images captured.

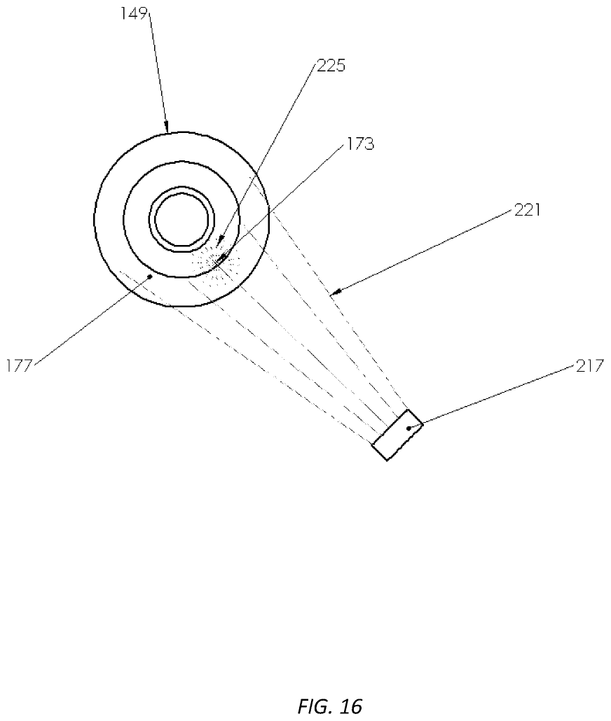

Referring to FIG. 16, in this embodiment, an infrared heat source (217) is used to flash heat to the sample. This “IR Probe” increases the contrast of contamination. This technique involves an exposure of infrared light (221) which may differentially heat the pharmaceutical and contamination (173) due to differences in the emissivity, heat capacitance, and thermal conductivity. The heat pulse duration will be between 1 ms to 500 ms. The IR heat (221) may be focused with a lens.

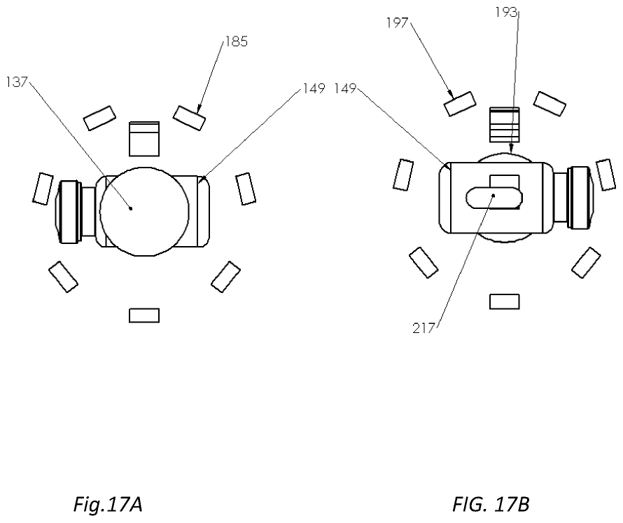

FIGS. 17A and 17B show heating of the sample may be accomplished using a single source (217) or multiple sources of infrared heat arranged in various configurations (185, 197) around the sample. The sources may flash simultaneously, independently, or in a sequence. The time between exposures may be controlled. The use of a camera (137) (193) will be used to capture the image. The camera could be a SWIR, NIR, UV, IR or regular light camera. The camera images could be shot independently or in sequence with the IR Probe.

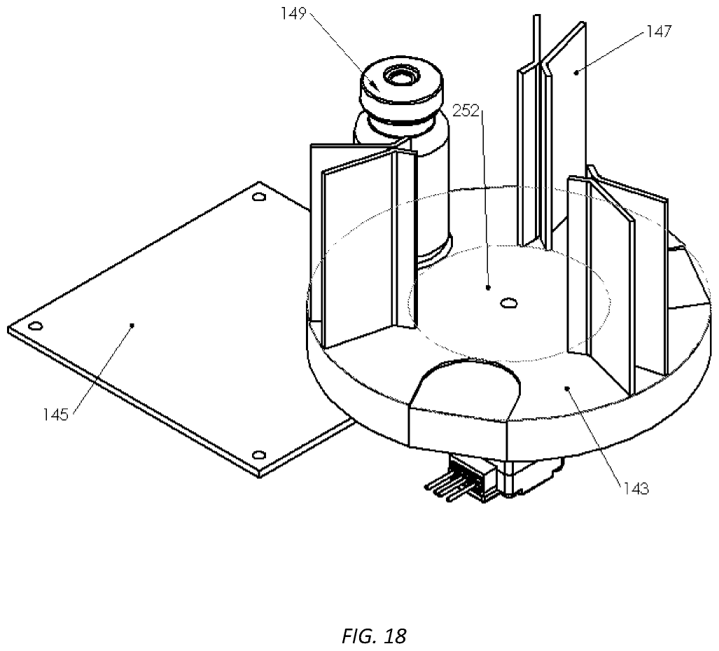

Referring to FIG. 18 various embodiments will be using a resonance system to vibrate with increasing amplitudes at various frequencies to cause excitation in the system. This excitation can then be tracked for differential motion inside the vial. The resonance system will be mounted below or on the side of the containers. This may be used in conjunction with other systems to better detect contamination. There will be a resonance plate (252) and a housing (255), mounting (143).

Detailed Example of a Process Using a System as Disclosed Herein:
1. A vial containing lyophilized pharmaceutical is placed into the inspection chamber in a vertical, angular or horizontal orientation.
2. Vial contains a septum and is sealed and sterile
3. Vial measures 25 mm in height and 12 mm at the OD.
4. Lyophilized powder is about 10% of the total volume of vial. (Range is 5% to 20%)
5. Door is closed and operator ensures settings correspond to product.
6. Operator initiates test sequence.
7. Machine exposes the vial to infrared energy simultaneously from three sources for 250 ms.
8. Within 100 ms of IR exposure, the machine takes 3 pictures simultaneously, using an IR camera, a visible light camera, and a UV camera. All cameras are using reflected light.
   a. Spectral response of the IR camera is 0.9 micron to 1.7 micron
   b. UV camera images light between 335 and 365 nm using a filter.
   c. Visible light camera is filtered against IR and UV wavelengths
9. An aperture is used to eliminate stray light from entering the camera detectors.
10. Images are magnified 2× with glass optics to enable higher resolution.
11. All images are captured digitally
12. The resonance system is activated.
13. The Heat strobe is also activated, and real time imaging is used.
14. The excitation of contents is tracked with imaging and evaluated.
15. Each of the images is compared against a data base of good and bad corresponding the frequencies at which they are captured.
16. The UV image is then subtracted from the visible image and compared to a corresponding data base of good and bad.
17. The IR image is then subtracted from the visible image and compared to a corresponding data base of good and bad.
18. If any of the images are found to have contamination, a red light shows on a display console and the sample attributes and images are captured. Operator removes vial and places it in a reject location with appropriate labeling.
19. If no images are found to have contamination, a green light shows on a display console and the sample attributes and images are captured. Operator removes vial and places it in an approved location with appropriate labeling.

The embodiments described herein are exemplary. Modifications, rearrangements, substitute materials, alternative elements, etc. may be made to these embodiments and still be encompassed within the teachings set forth herein.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices and components illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

1. An automated Process for detecting contaminants in a vial of pharmaceuticals, comprising;
imaging of at least one container loaded into an optically viewable element;
exposing the container to infrared energy;
acquiring images derived from the imaging, from at least two wavelength regions, the wavelength regions including Visible, Near infrared, Short Wave Infrared, and Ultraviolet;
agitating at least one container, at least one of before, during, or between image acquisition;
comparing the images from the different wavelength regions for contrast differences related to the presence of contaminants.

2. A system for detecting contaminants in a vial of pharmaceuticals, comprising;
at least one optically viewable vial holding element;
at least one infrared energy source;
at least two imagers, operating at different wavelength regions;
at least one container agitator; and,
at least one processor, the system configured to;
image at least one container loaded into the optically viewable holding element;
acquire visible images derived from the imaging, from at least two wavelength regions, the wavelength regions including Visible, Near infrared, Short Wave Infrared, and Ultraviolet;
agitate the at least one vial, at least one of before, during, or between image acquisition;
compare the images from the different wavelength regions for contrast differences related to the presence of contaminants, wherein; the at least one processor controls the imaging, agitation, and comparing operations.
